(19) United States
(12) Patent Application Publication
(10) Pub. No.: US 2004/0234995 A1
(43) Pub. Date: Nov. 25, 2004

(54) System and method for storage and analysis of gene expression data

(76) Inventors: Eleanor M. Musick, Germantown, MD (US); Dough Dolginow, Potomac, MD (US); Michael Elashoff, Germantown, MD (US); Da Wei Huang, Gaitherburg, MD (US); Supriya Menezes, Germantown, MD (US); Larry Mertz, Rockville, MD (US); Ramgopal Nadimpalli, Gaithersburg, MD (US)

Correspondence Address: PROCOPIO, CORY, HARGREAVES & SAVITCH LLP, 530 B STREET, SUITE 2100, SAN DIEGO, CA, 92101, US

(21) Appl. No.: 10/495,100

(22) PCT Filed: Nov. 4, 2002
(86) PCT No.: PCT/US02/35454

Related U.S. Application Data
(60) Provisional application No. 60/331,182, filed on Nov. 9, 2001.
(60) Provisional application No. 60/388,745, filed on Jun. 17, 2002.
(60) Provisional application No. 60/390,608, filed on Jun. 21, 2002.
(60) Provisional application No. 60/412,156, filed on Sep. 19, 2002.

(51) Int. Cl. C12Q 1/68; G06F 19/00; G01N 33/48; G01N 33/50

(52) U.S. Cl. 435/6; 702/20

(57) ABSTRACT

In a computer system for analysis of gene expression data, a gene expression database is organized in a hierarchical b-tree according to the descriptive and clinical sample attributes stored in the database. A user submits a query for searching the database and defines attributes on which to filter for each level of the b-tree. A simple search can be employed to arbitrarily group together leaf nodes depending on their attributes. The grouped leaf nodes are used as “control” and “experimental” sample sets. A t-test can be performed to test for statistically significant regulation between the control and experimental sample sets. In one embodiment, the results of the b-tree analysis are organized as a table of information which may be part of a relational database. The data in the database are encoded according to a three-state scheme based on regulation behavior. A similarity search algorithm can be performed on the encoded data to identify genes or gene fragments that show regulation profiles similar to the query gene or gene fragment, ranking the genes according to the level of similarity.

### RELATED APPLICATIONS

This application claims priority to U.S. Provisional Applications Ser. No. 60/331,182, filed Nov. 9, 2001, Ser. No. 60/388,745, filed Jun. 17, 2002, and Ser. No. 60/390,608, filed Jun. 21, 2002, each entitled “An Automated Computer-Based Algorithm for Organizing and Mining Gene Data Derived from Biological Samples with Complex Clinical Attributes”, and Ser. No. 60/412,156, filed Sep. 19, 2002, entitled “Comparative Gene Expression Database and Algorithm for Generating Same”, each of which is incorporated herein by reference in its entirety.

### BACKGROUND OF THE INVENTION

1. Field of the Invention

The present invention relates generally to systems and methods for organizing gene expression, gene annotation, and sample information in a relational format supporting efficient exploration and analysis. More particularly the invention relates to a system and method for automatically generating biologically-related sample sets, curating such sets by employing various quality control measurements and parameters, and using such sets for large-scale analysis and data mining of gene expression data.

2. Description of the Related Art

DNA microarrays are glass microslides or nylon membranes containing DNA samples (e.g., genomic DNA, cDNA, or oligonucleotides) in an ordered two-dimensional matrix. DNA microarrays, which commonly employ oligonucleotides or amplified portions of cDNA clones as probes, can be used to analyze gene expression. The DNA used to create a microarray is often from a group of related genes such as those expressed in a particular tissue, during a certain developmental stage, in certain pathways, or after treatment with drugs or other agents. Expression of that group of genes is quantified by measuring the hybridization of fluorescently-labeled RNA or DNA to the microarray-linked DNA sequences. By profiling gene expression, transcriptional changes can be monitored through organ and tissue development, microbiological infection, and tumor formation.

Also known as biochips, DNA microarrays can be created by linking monomeric nucleotides on the glass surface to make oligonucleotides. Another methodology, popular for making arrays of PCR products and organismal genes, uses robotic instruments to spot thousands of DNA samples onto a surface. This high-throughput approach increases reproducibility and production.

Devices and computer systems have been developed for collecting information about gene expression or expressed sequence tag (EST) expression in large numbers of tissue samples. For example, PCT application WO92/10588, incorporated herein by reference for all purposes, describes techniques for sequencing or sequence checking nucleic acids and other materials. Probes for performing these operations may be formed in arrays according to the methods of, for example, the techniques disclosed in U.S. Pat. No. 5,143,854 and U.S. Pat. No. 5,571,639, both incorporated herein by reference for all purposes.

Computer-aided techniques for monitoring gene expression using such arrays of probes have been developed as disclosed in EP Pub. No. 0848067, PCT publication No. WO 97/10365, and U.S. Pat. No. 6,308,170, the contents of which are herein incorporated by reference. Many disease states are characterized by differences in the expression levels of various genes either through changes in the copy number of the genetic DNA or through changes in levels of transcription (e.g., through control of initiation, provision of RNA precursors, RNA processing, etc.) of particular genes. For example, losses and gains of genetic material play an important role in malignant transformation and progression. Furthermore, changes in the expression (transcription) levels of particular genes (e.g., oncogenes or tumor suppressors), serve as signposts for the presence and progression of various cancers.

Using current DNA microarray technology, one can easily collect large amounts of data to indicate which genes or ESTs are regulated upwards or downwards during various disease states, following various pharmacological treatments, or following exposure to a variety of toxicological insults. However, while the quantity of data that one can gather with these techniques is very large, it is often out of context. The relevance of gene expression data is often determined by its relationship to other information within the context of the current analysis. For example, knowing that there is an increased expression of a particular gene during the course of a disease is important information. In addition, there is a need to correlate this data with various types of clinical data, for example, a patient's age, sex, weight, stage of clinical development, stage of disease progression etc. What is needed in the field is a way to correlate the vast amounts of gene and EST expression data that one can obtain using DNA microarrays with the corresponding clinical data from the samples that are tested.

Information on expression of genes or expressed sequence tags (ESTs) may be collected on a large scale in many ways, including the probe array techniques described above. One of the objectives in collecting this information is the identification of genes or ESTs whose expression is of particular importance. Researchers wish to answer questions such as: 1) which genes are expressed in cells of a malignant tumor but not expressed in either healthy tissue or tissue treated according to a particular regime; 2) which genes or ESTs are expressed in particular organs but not in others; and 3) which genes or ESTs are expressed in particular species but not in others.

Collecting vast amounts of expression data from large numbers of samples including all the tissue types mentioned above is but the first step in answering these questions. To derive full value from the investment made in collecting and storing expression data, one must be able to efficiently mine the data to find items of particular relevance. The need exists for an efficient and easy-to-use system for organization and analysis of complex gene, sample and expression relationships. It is necessary to study the behavior of genes in a holistic manner rather than individually because the expression of particular genes is neither isolated from nor independent from other genes. A better understanding of the complex network of gene interactions can lead to discovery of novel therapeutic mechanisms and new potential drug targets. For example, current sample-based analysis methods for gene expression data involve manual curation of sample sets. Investigators must begin an analysis with a specific goal (e.g., ‘today I will investigate Alzheimer's disease’) in mind and build their sample sets accordingly. This method biases the resulting analyses towards the initial goal of the investigator and leaves potentially interesting patterns undiscovered because the investigator did not have time to manually exhaust all potential analysis routes through the available data. To provide an example, discovering a gene regulated in Alzheimer's disease is interesting; finding a gene regulated across all known degenerative neural diseases is potentially far more useful.

Accordingly, the needs remains for a system and method for organization and analysis of complex gene, sample and expression relationships.

### BRIEF SUMMARY OF THE INVENTION

The system and method for analysis of gene expression data according to the present invention avoid the problems inherent in existing methods by allowing the user to define more general sample relationships in which he or she is interested and, thus, automate the creation of all possible valid sample sets defined by these general relationship parameters.

The aforementioned needs are addressed by providing methods and systems that correlate normal and diseased tissues or cell lines from humans and experimental animals with critical clinical findings improving target selection and prioritization. In addition, the system and method can be extended to correlate the effects of medication on tissue samples, for example, by comparing non-treated tissues versus treated tissues in a b-tree sorted by tissue and then by medication. In the same fashion, effects due to patient secondary diagnosis, age, race, gender and a myriad of lifestyle attributes (such as drug use, smoking, alcohol consumption, etc) and clinical diagnostic data (e.g., cholesterol levels, hematocrits, white blood cell counts, etc.) can be compared within the framework provided herein.

The system and method of the present invention provide the ability to examine the effects of therapeutic and prophylactic compounds on human and animal tissues or cell lines. One can easily study the mechanism of action of therapeutic compounds and the characteristics of experimental model systems by comparing the gene expression data with known therapeutic and experimental parameters. Similarly, the present system allows one to examine the affects of toxic compounds on tissues and cells in both a pre-clinical and clinical setting.

An efficient and easy-to-use query system and data analysis scheme for a gene expression data source is provided. The present system and method permit large scale gene expression databases to be fully exploited. This query system and data analysis method can be implemented in any one of a number of computational programming languages and processes known to those in the art. Using such a system, one can easily identify genes or ESTs (expressed sequence tags) whose expression correlates to particular tissue types. Various tissue types may correspond to different diseases, states of disease progression, organs, species, etc.

In an exemplary embodiment, the gene expression database is organized in a hierarchical b-tree according to the descriptive and clinical sample attributes stored. Other sources of data such as text files containing tabular sample data may also be similarly organized. As is known in the art, a b-tree is a generic data structure with properties that make it useful for database storage and indexing. B-trees use nodes with many branches, and records are stored in locations called “leaves.” The maximum number of branches per node provides the order of the tree. The b-tree algorithm minimizes the number of times a medium must be accessed to locate a desired record. The user defines attributes on which to filter for each level of the b-tree. The resulting leaf nodes of the tree then contain samples grouped according to the specifications of the user. A simple search grammar can then be employed to arbitrarily group together leaf nodes depending on their attributes. These grouped leaf nodes are used as “control” and “experimental” sample sets. A t-test, a well-known statistics procedure for testing for differences between two groups, is performed to test for statistically significant regulation between the control and experimental sample sets.

In one embodiment, the results of the b-tree analysis are provided as a table of information that can be stored in an electronic spreadsheet (e.g., a Microsoft® Excel® file), printed as a hardcopy or exported to commercially available data mining software tools such as Spotfire®, Partek® and others for data mining and visualization. This is particularly helpful for more complex data sets composed of several genes, gene families or entire pathways.

In an alternative embodiment, data corresponding to the control and experimental sample sets and comparisons between the sets can be used to construct a relational database of gene regulation events. This database can be used, for example, to assemble clusters for exploring relationships among a large number of different genes or disease states.

A number of distance calculation/clustering methods can be used for organization and analysis of gene expression data. These methods include hierarchical clustering, k-means (non-hierarchical) clustering, and self-organizing maps. Such methods assume that similarity measurements have been computed on continuous data rather than on discredited values. In addition, strictly Boolean (two-state) encoding can be used. However, because there is no real basis for selecting the “correct” clustering method, the different clustering algorithms can generate dramatically different results. As a result, determination of the “correct” interpretation depends solely on a priori biological knowledge. In a preferred embodiment, the system and method of the present invention employ a three-state encoding scheme which gathers qualitative conclusions from expression data based on qualitative methods rather than using the traditionally quantitative approaches. In this embodiment, data are preferably classified “+1” for upregulation and “−1” for downregulation, regardless of fold change value, and “0” for no change. Vectors are created corresponding to these encoded values, then a statistical method is applied to determine a level of similarity, i.e., a statistical distance, between any two probe sets. By calculating the level of similarity for all genes on a microarray, they can be ranked according to the similarity of their regulation profiles. In the preferred embodiment, a kappa statistic is used to provide the similarity measure for regulation profiles of various genes across different diseases and tissues.

According to a first aspect of the present invention, a method is provided in a computer system for hierarchically organizing information regarding biological samples using an n-order b-tree and a query grammar. The method includes: providing a data source including gene expression data derived from sample based analyses; defining relationships between data based upon descriptive and clinical sample attributes; comparing a control sample set against an experimental sample set with regard to the defined relationship; and displaying the results of such comparison. In a preferred embodiment this data source would be a relational database.

For example, if a user is interested in gene regulation in normal tissues when compared to disease states within that tissue the user can specify the tree sort order as “tissue/disease/morphology.” The leaf nodes then would contain samples sharing common tissue, disease state and morphology (a sample's appearance under a microscope) annotations. Each leaf node would then correspond to a set of samples one might normally construct manually.

Thus, for each tissue branch of the tree one can compare the morphologically normal sample set against sample sets for all diseases of that tissue in the database in parallel. This global comparison ensures that all genes showing significant regulation in the data in all possible disease processes are brought to light rather than only those genes regulated in the single area of initial interest to the investigator.

In another embodiment, a similarity search algorithm is provided for operating on global regulation profiles in gene expression data drawn from comparisons of normal and diseased tissue states. Known statistical and computational methods are combined with a data source of gene expression results to provide the user with valuable information, for example, identifying gene(s) that show regulation profiles similar to the query gene to, in turn, identify possible biological relationships to the query gene.

A further understanding of the nature and advantages of the inventions herein may be realized by reference to the remaining portions of the specification and the attached drawings.

### BRIEF DESCRIPTION OF THE DRAWINGS

FIG. 1 illustrates an example of a b-tree structure for a user-defined tree dividing samples by tissue, then disease, then morphology.

FIG. 2 provides a flow chart demonstrating the steps in the analysis process.

FIG. 3 provides an example of data generated from an outlier analysis detection and masking routine.

FIGS. 4a and 4b illustrate an example output from b-tree analysis in table form and the result of trinary (three-state) encoding of that b-tree output data, respectively.

FIG. 5 is a data model for a relational database of gene regulation events.

FIG. 6 is a flow diagram showing the analysis path for a three-state encoding scheme.

FIG. 7 is a table containing sample data encoded using the three-state encoded scheme.

FIG. 8 illustrates the comparison of two three-state encoded regulation strings (Gene1 and Gene2) by the kappa statistic.

FIG. 9 illustrates a sample output following analysis according to the present invention.

### DETAILED DESCRIPTION OF THE PREFERRED EMBODIMENTS

Microarray technologies enable the generation of vast amounts of gene expression data. Effective use of these technologies requires mechanisms to manage and explore large volumes of primary and derived (analyzed) gene expression data. Furthermore, the value of examining the biological meaning of the information is enhanced when set in the context of detailed biological sample profiles and gene annotation data. The format and interpretation of the data depend strongly on the underlying technology. Hence, exploring gene expression data requires mechanisms for integrating gene expression data across multiple platforms and with detailed sample and gene annotations. The present invention uses a hierarchical method for organizing biological samples for analysis using a b-tree and a query grammar to manage and explore gene expression and related data. The results of the b-tree analysis are organized in a relational database to permit data mining for identification of interrelationships between behavior of different genes or gene fragments, e.g., for one or more diseases, treatments, or demographics.

In a preferred embodiment of the present invention, this data is drawn from a relational database as an integrated product of three component databases that materialize the sample, gene annotation, and gene expression data spaces discussed in the previous section.

According to the present invention, a computer system is designed for hierarchically organizing information regarding biological samples using an n-order b-tree and a query grammar. The method includes: providing a data source including gene expression data derived from sample based analyses; defining relationships between data based upon descriptive and clinical sample attributes; comparing a control sample set against an experimental sample set with regard to the defined relationship; and displaying the results of such comparison. In one embodiment, this data source would be a relational database.

In a preferred embodiment, the present system and method are part of a combined database and data mining algorithm and system such as disclosed in co-pending applications Ser. No.09/862,424, filed May 23, 2001, Ser. No. 10/018,461, filed Dec. 19, 2001, and Ser. No. 10/094,144, filed Mar. 5, 2002. The disclosure of each application is incorporated herein by reference in its entirety.

The database and analytical engine preferably run on hardware from Sun Microsystems, Inc. (Palo Alto, Calif.) on the Solaris™ 8 Operating Environment (also from Sun Microsystems). The database is Oracle Server 8.1.7.3. Other software includes Visibroker® C++3.3.2 from Borland Software Corporation (Scotts Valley, Calif.), Java™ 2 SDK version 1.3.1.03 (available on the WWW from Sun Microsystems), Apache HTTP server 1.3.12 and Xerces-c 1.7.0 XML parser (both from Apache Software Foundation at www.apache.org), Expat 1.95.2 XML parser library (available from http://sourceforge.net), and Perl 5.6.0 and 5.6.1. For any of the identified software, later version may be used as well. Supporting documentation for the hardware and each of the listed software programs is incorporated herein by reference for purposes of this disclosure.

In an embodiment of the present invention, gene expression data may be generated using the Affymetrix GeneChip® platform, marketed by Affymetrix Corporation of Santa Clara, Calif., and may be represented in the Genetic Analysis Technology Consortium (“GATC”) relational format.

Samples may be associated with attributes that describe properties useful for gene expression analysis. For example, sample structural and morphological characteristics (e.g., organ site, diagnosis, disease, stage of disease, etc.) and donor data (e.g., demographic and clinical record for human donors, or strain, genetic modification, and treatment information for animal donors). Samples may also be involved in studies and therefore can be grouped into several time/treatment groups.

An example includes the DONOR table, which contains human donor attributes spanning various domains: general attributes such as HEIGHT, WEIGHT, RACE, DATE_OF_BIRTH; deceased fields such as DEATH_CAUSE, DEATH_AGE; sparse data fields such as exercise habits, diet profile, sleeping and smoking habits, alcohol and any recreation drug habits.

The sample attributes can be organized in classification hierarchies implemented using controlled vocabularies or existing taxonomies such as the Systematized Nomenclature of Medicine (“SNOMED”) topography and morphology axes, for sample organ and diagnosis, respectively.

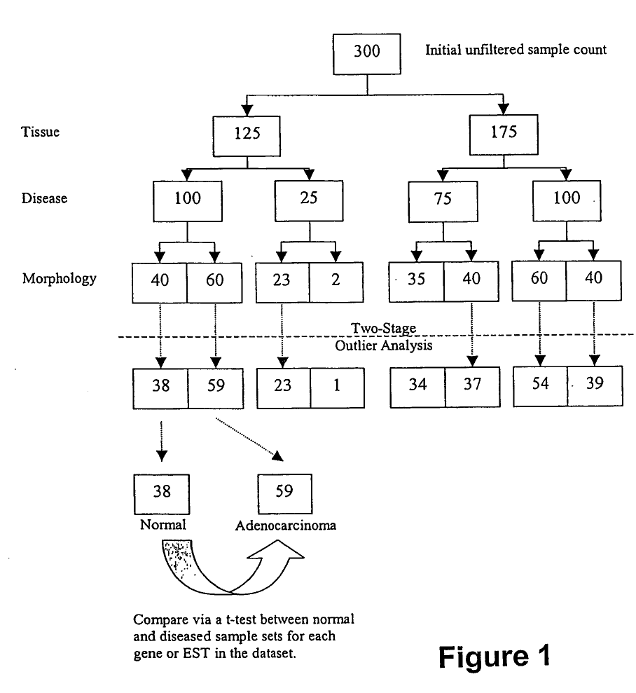

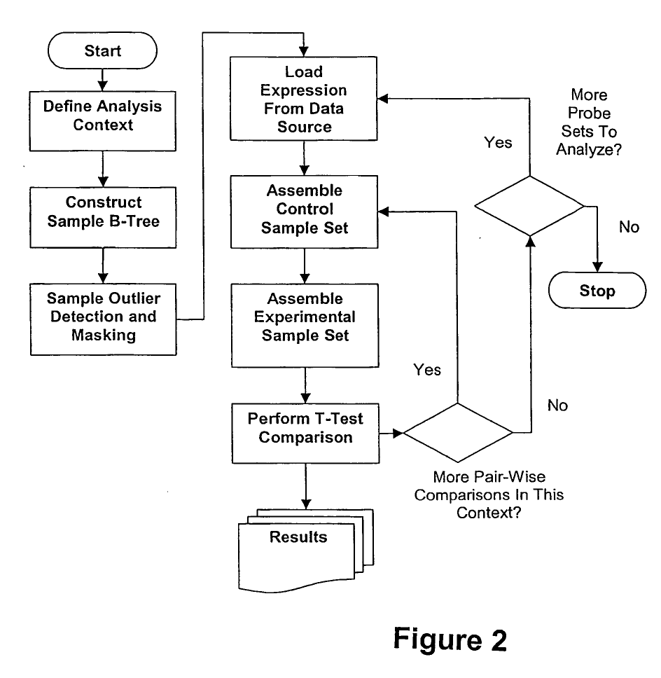

The hierarchical organization of samples is accomplished using an n-order b-tree, essentially a hash, i.e., associative array, of references to sub-hashes. Each level in the tree is hashed on the sample attribute the user assigned to that particular level. The value stored for each key is a reference to the hash representing that portion of the next level down in the tree. The leaf nodes of the tree contain a count of samples belonging to the final node rather than a reference to any further tree levels. An example of this structure is diagrammed in FIG. 1, whereas the schema describing the overall application of this invention in the definition and retrieval of data from a large scale gene expression data source is diagrammed in FIG. 2.

In FIG. 1, the example tree is shown having two distinct tissue types, each with two distinct diseases, each disease with two distinct morphologies. The numbers in each box represent exemplary sample counts at each level. The illustrated b-tree is provided as an example only and is not intended to be limiting. Actual trees generated from a large data source are typically much larger, having upwards of 40 to 50 individual tissue types with multiple diseases and morphologies per tissue.

The hierarchical tree serves to define the general characteristics of the sample space and the possible routes through the space to collect valid sample sets. This first characteristic is used by the system to determine the number and nature of possible pair-wise comparisons to be made. The second is useful in a first-pass evaluation of sample set size and subsequent “pruning” of the tree (to remove sample sets not meeting minimum size requirements) in order to reduce the number of comparisons performed to only those considered “valid”.

FIG. 2 illustrates the algorithm sequence for analysis according to the present invention. At “Start”, a user logs into the computer or computer network which links to the gene expression database and analytical engine. The user then enters his or her query, e.g., a specific gene or gene fragment to be searched, to “Define Analysis Context” and selects filtering criteria by defining attributes corresponding to each level of a b-tree, after which the system will “Construct Sample B-Tree”. Criteria for identifying and excluding outliers are entered in the “Sample Outlier Detection and Masking” step. Next, the system pulls expression data from the database (“Load Expression From Data Source”) for populating the b-tree. Using the retrieved data, the b-tree identifies and populates two sample sets in the steps of “Assemble Control Sample Set” and “Assemble Experimental Sample Set”. The identified sets are then compared for statistical significance in the step of “perform T-Test Comparison”. If more pair-wise comparisons need to be performed, e.g., there are different criteria are to be used to define “control” and “experimental” samples, additional control and experimental sets will be assembled. If no further pair-wise comparisons are needed, but more probe sets are available for analysis, the data for the additional probe sets may be loaded and the set assembly and comparison steps are repeated. After all data has been analyzed, the results are output to file or display means in a user interface in the “Results” step.

Auto-generated sample sets lack the level of curation possible in carefully annotated sets produced manually. In order to address this concern, the inventive system and method employ two additional levels of filtering to further curate and trim the samples and/or leaf nodes of the tree. These processes involve evaluation of various quality control parameters related to GeneChip® or other microarray data. Samples or sample sets not meeting certain QC criteria can be trimmed from the analysis to bolster the quality of gene expression data for comparison. This allows users of such a system the benefits usually afforded by manual curation without the costly time investment.

Parameters evaluated by this sample set generation method, in a preferred embodiment using Affymetrix GeneChip® data, include, but are not limited to, scale factor, raw-Q (a parameter indicating chip noise), the percentage of genes called present by Affymetrix algorithms, the percentage of saturated genes, and 5′/3′probe intensity ratios for the control genes GAPDH and β-actin. Within a sample set or leaf node of the tree, the mean or median value ±3σ (standard deviations) for each of these parameters can be calculated (for example). In addition, contributions of these parameters to the QC process can be differentially weighted depending on the inherent effect each has on the microarray gene expression data.

Using a binary encoding scheme, each sample is given a score for each of six parameters. If, for each sample, a parameter value falls within the designated range it would be assigned a value of “0”, whereas those parameters that fall outside the acceptable range would be assigned a value of “1” and would be labeled an “outlier” in that particular parameter.

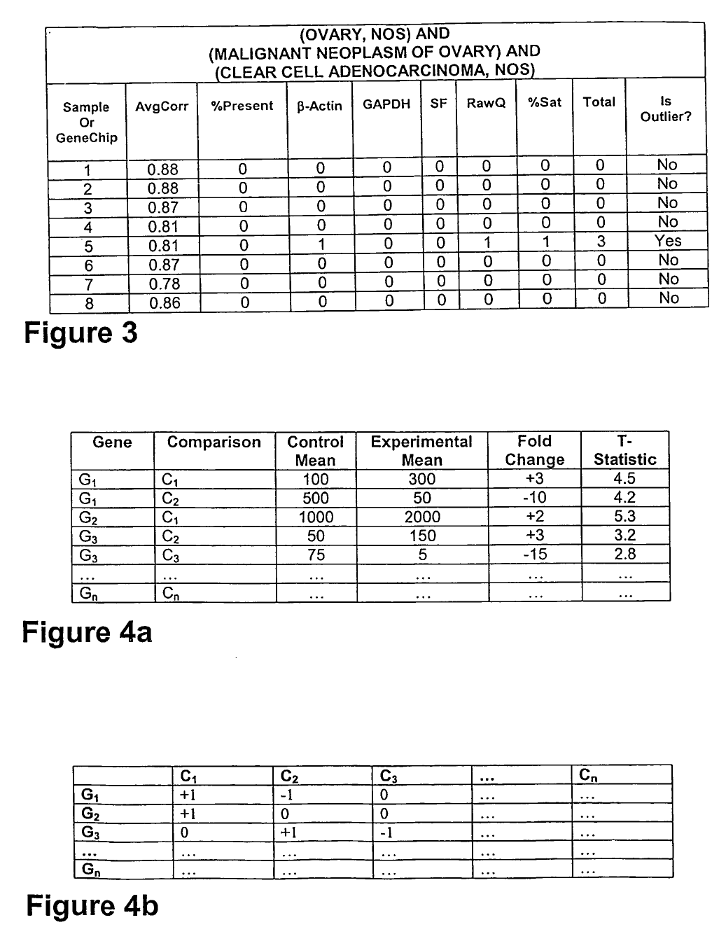

A matrix can be generated for each sample set node of the tree listing these binary values with rows named by sample/GeneChip® identifiers and columns named by parameter (see, e.g., FIG. 3). For each microarray, the number of failed parameters can be totaled and if this number reaches a certain pre-defined level, decisions can be made to remove the sample from further analysis. FIG. 3 illustrates a sample data table generated during chip parameter outlier analysis. The AvgCorr column indicates the average correlation calculated from each sample drawn from a correlation matrix. Sample 5 (see column 1) registered a value of “1” for each of the parameters β-Actin (column 4), RawQ (column 7), and % Sat (column 8) for a total of value of 3 (column 9). As a result, sample 5 was declared an “outlier” and removed from the sample set and from all downstream analysis. If there are multiple microarrays for a particular sample, such as when running a sample across the Affymetrix Hu95 and Hu133 GeneChip® microarray sets, a decision can be made to remove one or more microarrays from the analysis or even the entire sample, (composed of >1 microarrays) if a significant number of microarrays assigned to that sample fail to meet the predetermined QC criteria.

In addition to curation of tree-node sample sets based on microarray performance parameters, it may also be desirable to trim samples that exhibit deviant biological behavior with respect to overall within-cohort gene expression measurements and patterns of the nodal sample set. Such samples could be simply misclassified or improperly coded in the database, or may possess unique clinical parameters not taken into account by the user's current b-tree sort order, such as percent tumor infiltration or alternative systemic secondary diseases. For such biological analyses, an automated approach utilizing, but not limited to, principal component analysis (PCA), leave-one-out (LOO) analysis, or partial least squares (PLS) analysis can be implemented to augment the statistical accuracy of automated sample set creation and curation.

PCA is a data-reduction technique known in the art that provides for the reduction of high-dimensional data into so-called ‘principal components’. This technique is used within single sample sets to determine each sample's general similarity to other members within the group.

LOO analysis, which is also known in the art, is used to determine, between any two sample sets, which samples in either set would, when removed, have a disproportionate effect on the results of a t-test between the two sets.

PLS analysis, an extended multiple linear regression technique also known in the art, can also be used to determine, again between any pair of sample sets, which samples are most ‘unlike’ their supposed cohorts. This method differs from PCA in that in PLS samples ‘unlike’ their cohorts are defined as samples affecting the expression profile difference between the two sample sets rather than mere strict difference from within-set cohorts which may or may not have an effect on comparative gene expression.

Upon the discovery of additional clinical parameters that consistently contribute to altered large-scale gene expression behavior, these newly identified parameters can be incorporated into new tree sort orders to generate more accurate sample sets as described in this embodiment to create new gene analysis contexts.

Once a hierarchical b-tree has been constructed and pruned of invalid samples (whether by physical microarray parameter outlier analysis or biological outlier analysis, as discussed above) and sample sets (for sets containing too few samples for statistically rigorous results), analysis can begin. The system begins at the root node of the b-tree and runs a depth-first search, as illustrated in FIG. 1.

Another layer of complexity may be added to the b-tree analysis to attack the underlying biology inherent in this kind of sample organization. For each set of leaf nodes, one of two kinds of comparisons is useful and which type to select depends upon the attributes used in the b-tree sort order. For the normal versus disease state example given above (using a tree with sort order ‘tissue/disease/morphology’) the “normal” sample set is selected as a control and compared one at a time against each disease state (here, designated as the “experimental sample set”). This is termed a 1×1 comparison since a single leaf node is being compared against another single leaf node.

Alternative paths of analyses involve comparing some group of samples sharing a particular attribute with all other samples not sharing that attribute. This can be termed a 1×N comparison. As an example, one can examine medication effects by comparing ACE (angiotensin converting enzyme) inhibitor-treated cardiac samples from patients against similar tissue from patients not undergoing ACE inhibitor treatment (regardless of other treatments). Visually this can be represented in the tree by selecting the leaf node for ACE inhibitor-treated cardiac tissue as the experimental group and combining all other morphologically normal cardiac leaf nodes as the control group for a 2-level deep tree defined as ‘tissue/medication’.

A third type of comparison within the method of the present invention is also possible and will be referred to as an “N×N comparison”. An N×N comparison would involve taking all leaf nodes that share more than one attribute and comparing them against all leaf nodes that share the opposite of those attributes, producing control and experimental sample sets that both incorporate more than one individual leaf node.

These arbitrary leaf node groupings are defined by a simple search grammar implemented to compare attributes either based on text strings (for descriptive attributes) or bucketed numeric values (for numeric attributes e.g., patient age or cholesterol level). The search grammar consists of an array of references to sub-arrays, a maximum of one sub-array per level of the b-tree (and an implied minimum of no sub-arrays, which would return the entire body of samples). Each sub-array can contain one or more search terms (all of which are logically AND'd together). This array of arrays then acts like a filter, selecting which paths through the b-tree are valid in the current search context.

For example, in a tree built using a search order defined as ‘tissue/secondary patient diagnosis’ the term

[[T-62000],[DE-38010]]

specifies the branch containing all liver samples (T-62000 is the SNOMED code for liver) and the sub-branch containing liver samples from patients with hepatitis (DE-38010, the SNOMED code for hepatitis). This forms the experimental set. The control set, namely all liver samples from patients not infected with hepatitis, would be queried using

[[T-62000],[˜-DE-38010]].

The grammar defines a tilde (‘˜’) as the negation operator. Thus, the above operation collects all liver samples that come from patients that are not infected with hepatitis. The samples returned by this statement then form the control set.

For each gene in the analysis (usually on the order of 60,000+ genes (or gene fragments) in the case of the Affymetrix U95 GeneChip® array), the average difference values are retrieved for each sample in each set. Sample set means, medians and variances are then calculated.

The pair-wise comparison method used by this system is efficient and modular. A two-tailed t-test is performed on the means and variances of the control and experimental sample sets to determine the statistical significance of the separation between the two sample sets. The null hypothesis used for the t-test is that the population means for the logs of the expression values are the same in the two sample sets. The alternative hypothesis is that the means are different.

Fold change is calculated on a per-gene basis, i.e., the fold change algorithm is applied to each gene separately for each comparison. However, in order to perform a t-test, both sample sets must have more than one sample regardless of whether a fold change can be reported.

The result of the t-test is screened at an alpha value ranging from 0.05 to 0.001 and all genes meeting the selected criterion are output to a result table along with supporting statistical data. Alternative statistical methods may be used to determine significance of sample set mean separation since the system was designed to remain modular and statistically method-agnostic.

The hierarchical method for organizing biological samples for analysis using a b-tree and a query grammar can be implemented in system memory or, alternatively, can be implemented on a disk file and searched using b-tree file searching algorithms found in modern database design and implementation practices.

The current invention allows for AND'ing together search terms in the grammar, that is, one can create groups based on samples that are not one thing AND not another. It will be appreciated that this grammar can be extended to allow for a logical “OR” operator, e.g., group samples that are one thing OR another.

It should also be noted that the b-tree mentioned in the current invention can be extended and populated with genes instead of samples, building a tree to refine gene sets based on shared attributes (such as gene ontology, cross-species homology, functional domains, etc.). Combining selected leaf nodes from a sorted gene tree with selected leaf nodes from sorted sample trees offers a user very fine-grained control over analysis results (e.g., display all G-protein coupled receptors up- or down-regulated in any cancerous tissue).

In data creation and analysis applications where the expression metrics of more than one gene or gene fragment are being measured and studied, it may be desirable to examine the behavior of such subjects with respect to large scale biological applications. Such examples of multiple gene sets include members of gene or protein families such as G-protein coupled receptors (GPCRs), nuclear hormone receptors (NHRs) and protein kinases. Additional gene sets could encompass genes related by a biological process or pathway such as cell signaling transduction pathways, cell receptor-mediated secretory processes, apoptosis and cell death, cell division, etc. and other gene families and assemblies known to those in the art.

It is also conceivable that gene expression patterns for all genes within an organism could be created and compared to discover new relationships between genes based on functional genomics. One application could be for the categorization of unknown and unannotated genes and ESTs exhibiting behavior similar to that of well-characterized genes.

Furthermore, the present invention can be used to analyze data in a more traditional sample set-centric approach. Selecting a single control and experimental set of interest from a populated b-tree and iterating analyses for every gene across this single comparison would provide a global view of gene expression activity within a particular disease state or other biological context based on b-tree sort order.

In one embodiment, the gene expression results obtained from comprehensive b-tree comparisons for each gene (or gene fragment) are summarized in a matrix using a trinary, or similar, encoding scheme where up- and down-regulation of gene expression in the experimental (e.g. diseased tissue) state versus the control (e.g. normal tissue) would result in the assignment of 1 and −1, respectively, to the location i,j, where i represents the row in the matrix for a particular gene or gene fragment and j represents the column in the matrix for a particular pair-wise comparison (e.g. normal liver vs. liver cancer, etc). Thus, fold change values of the gene expression are not compared; rather, the qualitative aspect of gene regulation is used as the encoding scheme and as the basis for comparison.

It follows that a value of “0” would then be applied to ij locations where no gene expression regulation was observed for the i^{th} gene or EST within the j^{th} comparison. One can envision the application of this or a similar encoding scheme across the b-tree comparison space for each gene or gene fragment contained on a GeneChip® chip set or similar microarray.

This matrix can then be bi-directionally clustered to group together genes or gene fragments, or disease states, that are behaving in a similar manner across multiple related and divergent human diseases and clinical contexts within a gene expression database. This technique is useful for learning the structure of a dataset without making any preconceived assumptions about the structure.

The length of the bit string generated per gene would be equal to the number of comparisons gathered from the b-tree (whose size, in turn, depends on the variety and depth of samples pulled from the initial data source). Pattern searching algorithms can be applied to the clustered matrix to discover genes and gene fragments that exhibit predictably similar or, also just as interesting, predictably opposing gene expression regulation patterns in multiple experimental states.

FIG. 4 provides an example of the trinary (three-state) encoding scheme for downstream clustering of gene regulations derived from the algorithmic b-tree analysis. In FIG. 4a, exemplary output from the b-tree analysis algorithm is shown arranged in tabular form. This is the initial data from which the trinary encoding scheme will be derived. Entries of the form Gₓ, represent probe sets on a microarray, e.g., a GeneChip®, representing a particular gene. Table entries of the form Cₓ indicate pair-wise comparisons, e.g., normal brain tissue compared to that of patients suffering from Alzheimer's disease. Numeric entries are for illustrative purposes only, and mean values are given in unitless “average difference” intensity values. FIG. 4b is a table showing the data from FIG. 4a encoded using the trinary encoding scheme. In an alternate embodiment, an Eisen-like color-coding scheme can be applied to this data table to facilitate analysis. For example, the +1 cells can be red and the −1 cells can be green. Clustering algorithms known in the art can be applied to cluster genes and disease states that share similar, or predictably dissimilar, expression profiles.

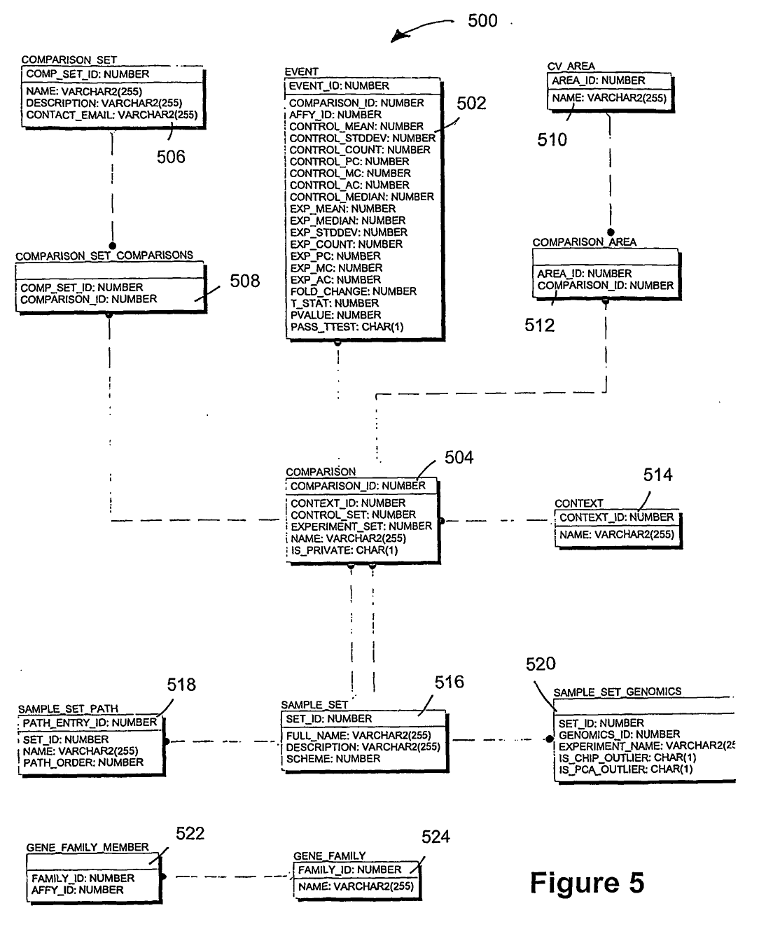

In an alternate embodiment, the gene expression results obtained from comprehensive b-tree comparisons for each gene or gene fragment are summarized in a group of relational tables within a relational database 500, as shown in the data model of FIG. 5. Each block in the diagram represents a relational table, with the relationships between the tables indicated by dashed lines between the tables. “Event” table 502 contains the results of regulation events, identification information for each control and experimental sample, results of the comparison of the control and experimental sample sets, e.g., fold change analysis, t-test, etc., and identifiers for each comparison. The primary key for Event table 502 is a unique identifier for each regulation event: EVENT_ID: NUMBER.

The table designated “CV_Area” 510 contains control vocabulary which may be used to narrow the area in which an analysis is conducted. For example, a search can be limited to information relating to the central nervous system or cardiovascular system. The primary key in this table is an AREA_ID: NUMBER that is associated with the name of the different possible areas of interest.

The “Comparison” table 504 contains records to describe the nature of the comparison and includes two foreign keys for identification of the control sample set and experiment sample set. The primary key in this table is the “COMPARISON_ID: NUMBER”, a unique identifier assigned to each comparison between a control sample and an experimental sample. CONTEXT_ID: NUMBER corresponds to “Context” table 514, which provides a description of how the b-tree that produced the comparison result was organized. For example, referring to the example of FIG. 1, the b-tree sorts the sample set based on organ, disease, and morphology, respectively.

The “Comparison_Area” table 512 is a joined table which links area information from table 510 with comparison information contained in table 504.

“Comparison_Set” table 506 contains information for comparison data corresponding to pre-constructed sample sets that were generated independent of the b-tree analysis. Typically, such sample sets possess some error which prevents automated analysis, e.g., incorrect annotation. Such sample sets are annotated manually by investigators whose contact information is provided in the table along with brief descriptions of the sample set(s). The primary key, COMP_SET_ID: NUMBER, provides a unique identifier to the pre-constructed sample sets.

“Comparison_Set_Comparisons table 508 is a joined table combining the identifiers for the automatically-generated comparisons from b-tree analysis, from table 504, and manually-generated comparisons from table 506.

“Sample_Set_Path” table 518 contains records of the pathway that was followed to navigate the b-tree to arrive at the leaf node which corresponds to the sample set. The primary key in this table is the PATH_ENTRY_ID: NUMBER.

Sample_Set” table 516 contains records of the names and descriptions of the final sample sets generated by the b-tree analysis. The primary key is SET_ID: NUMBER, a unique number assigned to each sample set.

“Sample_Set_Genomnics” table 520 is a joined table linking the unique set identifier with a series of numerical identifiers which are foreign keys pointing to a table in the database that defines the SAMPLE object.

“Gene_Family” table 524 provides information about the gene family within which the probe set on an Affymetrix® microarray might fall, including the gene family name. For example, there are approximately 500 probes on the Affymetrix® U133 GeneChip® microarray that qualify as GPCRs, so Gene_Family table 524 would have an entry for GPCRs. The primary key for this table is the FAMILY_ID: NUMBER.

“Gene_Family_Member” table 522 is a joined table linking the FAMILY_ID: NUMBER from table 524 with the identifier assigned to each gene or gene fragment according to the Affymetrix® identification system, e.g., probe set numbers and chip identification number. The AFFY_ID:NUMBER is recorded in Event table 502. For the example cited in the preceding paragraph, “Gene_Family_Member” table 522 would have approximately 500 entries, each with a foreign key, AFFY_ID, pointing to a table in the database that defines the AFFY_FRAGMENT object. This organization is helpful for parsing events into gene-family specific groupings, for example, to find all GPCRs that are regulated in kidney cancer.

The relational database can be used to rapidly access and compare gene expression data generated for every gene or gene fragment on one or more GeneChip® microarrays, or other types of microarrays, thus providing for analysis of very large volumes of data to identify patterns and interrelationships between, e.g, diseases, treatments, etc. It may be appropriate to compare gene expression data for every gene fragment in a microarray with that of every other fragment on the same microarray. The resulting comparison data can be clustered according to any of a number of desired parameters, for example, normal versus disease, organ type, demographics, etc., then printed out in a report form. The database, which will be quite large, should preferably be refreshed on a regular basis in order to include new comparisons that become available as a result of ongoing research, thus expanding the possibility of identifying new patterns between gene regulation and diseases, organs, treatments, etc.

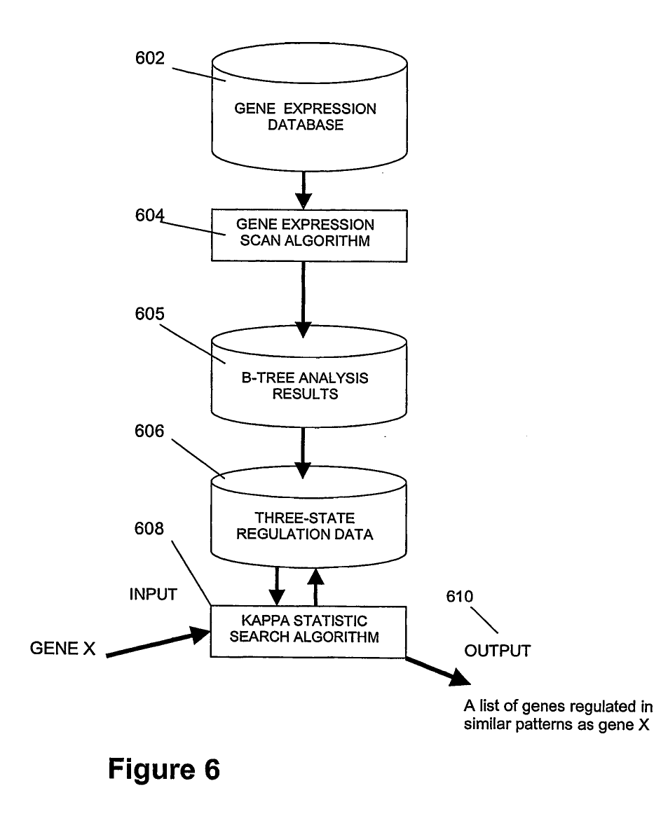

FIG. 6 illustrates an embodiment of the invention in which the three-state encoding scheme is used in conjunction with a statistical comparison method that provides a measure of similarity between any two probe sets. The gene expression database 602 and the gene expression scan algorithm 604 based on the hierarchical b-tree analysis have been previously described. (The gene expression scan algorithm 604 is shown in the flowchart of FIG. 2 and uses a b-tree analysis to generate the relational database 500 of FIG. 5, which in FIG. 6 is identified as b-tree analysis results 605.) Following encoding of the data according to the three-state scheme, database 606 is created and stored. In the preferred embodiment, because these values can be reused, the three-state encoding of the entire gene expression database 602 is performed in advance of any search query then stored in database 606. Upon input of a search query for a gene or gene fragment of interest (designated here as “Gene X”), algorithm 608, which in the preferred embodiment uses the kappa statistic, compares the trinary-encoded regulation data in database 606 to determine the level of similarity in gene regulation profiles relative to Gene X, generating output 610 in the form of a list of genes which are regulated in patterns similar to those observed for the gene or gene fragment of interest.

In a preferred embodiment, gene expression database 602 is a comprehensive collection of normal and diseased gene expression data. The sources of data in database 602 can be proprietary sources or publicly-available databases which may be used for data mining by pharmaceutical, biotechnology and other researchers and clinicians. For example, the databases described in previously-reference applications Ser. No. 09/862,424, No. 10/018,461 and Ser. No. 10/094,144 may be used. A preferred database is the GX™ Data Warehouse which is part of the Genesis Enterprise System™ offered by Gene Logic Inc. (Gaithersburg, Md). The expression regulation behavior is aggregated into discrete three-state values, e.g., +1, −1 and 0, based on the direction of fold change values in normal versus disease comparisons. It should be noted that the three-state encoding scheme can use any combination of three indicia for designating the direction of regulation. Thus, while the exemplary embodiment is described herein in terms of +1, −1 or 0, an alternate encoding scheme could assign symbols such as alphabetic or alphanumeric characters or combinations of characters to indicate direction. For example, “U”, “up”, “P”, “plus”, or “>” could be used to indicate up-regulation, “D”, “down”, “M”, “minus” or “<” could be used to indicate down-regulation, and “N”, “none”, “NC”, “no change”, or “=” could be used to indicate no change. Other variations will be readily apparent to those in the art.

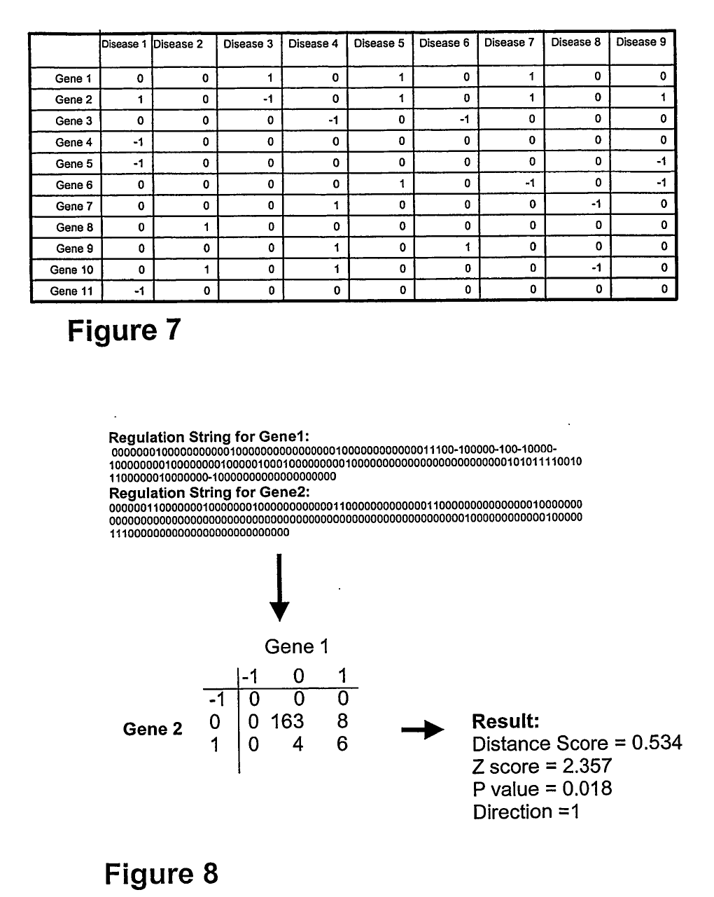

For example, if Gene X is up-regulated 3.1 fold in breast cancer, the assigned value for Gene X in a database for breast cancer would be +1. If the same gene is down-regulated with a fold change of −2.5 in liver cancer, it would be entered in the database for liver cancer as −1. An example of the data format for a number of different genes (Genes 1 through 10) and different diseases (Diseases 1-9) is shown in FIG. 7. For each gene in database 602 there will be stored a corresponding three-state vector of length N representing that gene's regulation pattern in the N tissue/disease state combinations available in the database.

The kappa statistic is a method of quantifying the level of agreement between two vectors of values. It enables the comparison of observed agreement versus agreement expected merely by chance. The agreement is quantified as an “agreement distance score” which is between zero, when agreement is no better than chance, and one, when there is perfect agreement. The formula for the kappa statistic (κ) is:

$$κ = \frac{{p}_{o} - {p}_{e}}{1 - {p}_{e}}$$

where pₒ is the probability of observed occurrence and pₑ is the probability of expected or chance agreement. The standard error of the kappa statistic is given by

$$s e ( κ ) = \sqrt{\frac{{p}_{e}}{N \cdot ( 1 - {p}_{e} )}} .$$

The Z score (the measure of statistical significance) is (κ/se(κ)).

For a given gene, its regulation vector is retrieved from the data source described above and compared, using the kappa statistic, to measure the distance between the gene and every other gene in the data source. FIG. 8 illustrates the hypothetical regulation strings for Gene1 and Gene2 then creates a matrix of the three-state vectors for these two strings. The results of the kappa statistical analysis are shown in the figure as distance score along with the Z score, the associated P value (the probability that the null hypothesis is true, calculated from the Z score) and direction. A list of high scoring genes is then generated.

In an alternate embodiment, other distance metric calculations can be employed in place of the kappa statistic. For example, score systems based on raw correlation coefficients or Euclidean distance can be used.

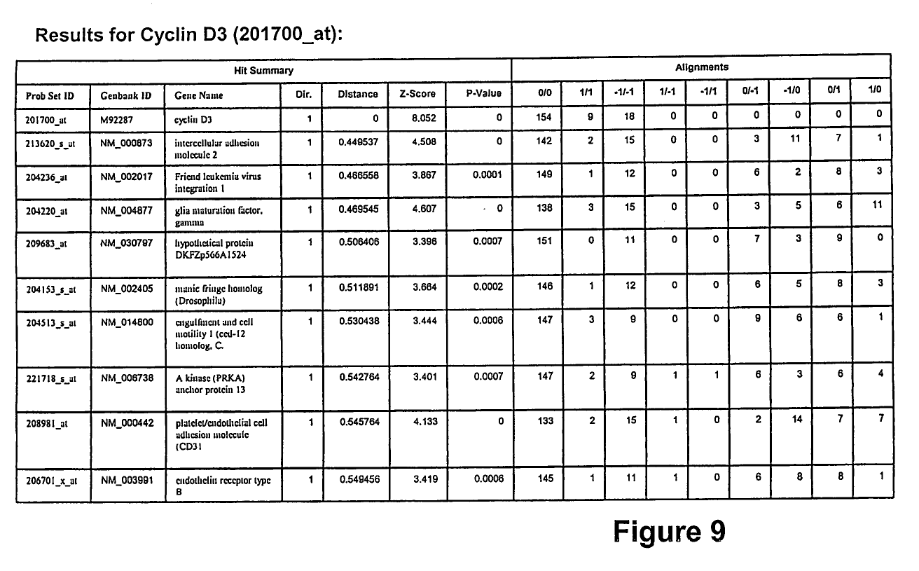

A user can query the data stored in the data source, e.g., from a chip-wide scan, in a piecemeal fashion, retrieving a list of co-regulated genes with the statistics described above. The output format should be readily understood and interpreted by the research community at large, particularly when compared to the dendograms and complex tree-based visualization used with many existing programs. An example of the output produced according to the present embodiment is shown in FIG. 9, which provides results from a search for cyclin D3. The table lists the top ranked hits for similarity (distance score, in order of increasing distance) based on the kappa statistic and includes information such as Affymetrix probe set ID, Genbank ID (or other external database), gene name, the values obtained from kappa statistic analysis and the alignments, i.e., the vector length N for the different pairs of three-state values corresponding to the N tissue/disease state combinations available in the database.

In the preferred embodiment, the algorithm for searching and extracting three-state encoded data, performing the pairwise similarity evaluation using the kappa statistic and generating output was implemented in Perl and S-Plus® (Insightful Corporation; www.insightful.com). As will be apparent to those of skill in the art, other programming languages and software may be used to perform some or all of the steps of the algorithm. For example, the statistical package available from SPSS, Inc. (Chicago, Ill.; www.spss.com) can be used for performing the kappa statistical analysis and generating the various values of interest. Similar statistical software is available from SAS Institute, Inc. (Cary, N.C.; www.sas.com).

The three-state encoding of gene regulation data according to the present invention provides a novel way to view expression data. The three-state values represent regulation directionality and are more logical from a biological standpoint. In contrast, two-state (Boolean) values are based on whether a gene is present or not, or whether the gene is regulated or not, without regard to directionality. Existing continuous analysis approaches use average mean expression values which can contribute a significant amount of noise to downstream clustering attempts. The three-state values of the present invention augment the ability to determine consistent gene behavior across states. For example, if two genes are primarily +1, −1 or −1, +1, then an instance in which they are +1, +1 can be considered a negative result. Boolean techniques, on the other hand, would be unable to identify such a detail.

In the foregoing specification, the invention has been described with reference to specific exemplary embodiments thereof. It will, however, be evident that various modifications and changes may be made thereunto without departing from the broader spirit and scope of the invention as set forth in the appended claims and their fill scope of equivalents.

What is claimed is:

1. A method for analyzing gene expression data, the method comprising:
(a) organizing data pertaining to a plurality of samples into a b-tree comprising a plurality of levels, each level comprising a plurality of leaf nodes;
(b) defining a plurality of attributes for filtering the data at each level of the b-tree;
(c) distributing the data among the plurality of leaf nodes according to the plurality of attributes;
(d) grouping the leaf nodes according to their corresponding attributes;
(e) defining a control sample set and an experimental sample set;
(f) performing a t-test comparing the experimental sample set with the control sample set; and
(g) generating a table of t-test results.

2. The method of claim 1, wherein the t-test results and information for identifying the gene expression data are stored in a relational database.

3. The method of claim 2, wherein the relational database comprises a plurality of relational tables comprising:
the table of t-test results; and
at least one second table of the plurality of relational tables comprising a plurality of labels for associating the plurality of t-test results with descriptions of the comparisons to which they correspond.

4. The method of claim 1, further comprising:
defining a different control sample set and a different experimental sample set;
repeating steps (e) and (f) for a plurality of pairwise comparisons; and
entering the results of each step (f) into the table.

5. The method of claim 1, wherein the plurality of attributes comprise structural and morphological characteristics of gene expression data.

6. The method of claim 1, wherein the plurality of attributes is selected from the group consisting of organ site, diagnosis, disease, stage of disease, demographic and donor data.

7. The method of claim 6, wherein donor data is from a human donor and is selected from the group consisting of height, weight, race, date of birth, cause of death, age at death, exercise habits, diet profile, sleeping habits, smoking habits, alcohol habits, and drug habits.

8. The method of claim 6, wherein donor data is from an animal donor and is selected from the group consisting of strain, genetic modification and treatment information.

9. The method of claim 1, further comprising filtering the data to prune data sets that are smaller than a pre-determined minimum sample size.

10. The method of claim 1, further comprising filtering the data to prune outlier samples.

11. The method of claim 1, further comprising filtering the data to prune data sets that fail to meet pre-determined quality control criteria.

12. The method of claim 1, wherein step (d) comprises performing a simple search to compare attributes by using a search grammar comprising an array of references to sub-arrays, each sub-array comprising at least one search term.

13. The method of claim 1, wherein the t-test results are encoded according to a three-state scheme where up-regulation relative to the control sample is assigned a first symbol, down-regulation relative to the control sample is assigned a second symbol different from the first symbol and no change relative to the control sample is assigned a third symbol different from the first and second symbols.

14. The method of claim 13, wherein the first symbol is +1, the second symbol is −1, and the third symbol is 0.

15. The method of claim 13, further comprising operating on the encoded t-test results using a similarity search algorithm to measure a level of similarity between a plurality of different experimental sample sets.

16. The method of claim 15, wherein the similarity search algorithm comprises the kappa statistic.

17. The method of claim 15, further comprising ranking the encoded t-test results according to the level of similarity.

18. A computerized storage and retrieval system of biological information comprising:
a stored database containing records pertaining to a plurality of gene regulation events for each of a plurality of control samples and experimental samples, wherein the database comprises a plurality of relational tables, each relational table having a means for linking to at least one other relational table of the plurality; the plurality of relational tables comprising:
a first table of the plurality of relational tables comprising a plurality of gene regulation event categories into which at least some of gene regulation events are grouped, the first table comprising results of a plurality of comparisons of selected control samples and selected experimental samples, wherein the selected control samples and selected experimental samples are results of a b-tree analysis;
at least one second table of the plurality of relational tables comprising a plurality of labels for associating the plurality of comparisons with descriptions of the comparisons, and a user interface allowing a user to selectively view information regarding the plurality of gene regulation events.

19. The system of claim 18, further comprising a third table of the plurality of relational tables comprising one or more manually-generated comparisons.

20. The system of claim 18, wherein the plurality of relational tables further comprises one or more tables selected from the group consisting of a control vocabulary table for selecting one or more area of analysis, a comparison table for describing a nature of each comparison, a context table for describing the organization of the b-tree, a sample set path table describing a pathway used to navigate the b-tree, and a gene family table for describing a gene type within which the sample sets fall.

21. The system of claim 18, further comprising a database for storing encoded data, wherein the encoded data comprises the results of the b-tree analysis compared by performing a t-test then encoded according to a three-state scheme where up-regulation in the selected experimental sample relative to the selected control sample is assigned a first symbol, down-regulation is assigned a second symbol different from the first symbol and no change is assigned a third symbol different from the first and second symbols.

22. The system of claim 21, wherein the first symbol is +1, the second symbol is −1, and the third symbol is 0.

23. The system of claim 21, further comprising a similarity search algorithm for operating on the encoded data to measure a level of similarity between a plurality of different experimental sample sets.

24. The system of claim 23, wherein the similarity search algorithm comprises the kappa statistic.

25. The system of claim 23, wherein the user interface outputs a report comprising a ranking the encoded data according to the level of similarity.

26. A database system having a plurality of internal records, the database comprising:
a first plurality of records specifying gene regulation events for a plurality of samples;
a second plurality of records specifying comparison results from comparison sets comprising selected experiment sample sets and selected control sample sets, wherein a first portion of the plurality of samples is designated sample control sets and a second portion of the plurality of samples is designated experiment sample sets, wherein the comparison results are derived from b-tree analysis of the comparison sets;
a third plurality of records specifying comparison context comprising data describing how a comparison set was selected and analyzed; and
a fourth plurality of records comprising a plurality of links for associating the first, second and third plurality of records.

27. The database of claim 26, wherein the statistical analysis of the comparison sets comprises a t-test.

28. The database of claim 27, further comprising a plurality of encoded records wherein the comparison results are encoded in a three-state scheme corresponding to up-regulation, down-regulation and no change.

29. The database of claim 28, wherein the encoded records are acted on by a similarity search algorithm.

30. The database of claim 29, wherein the similarity search algorithm is the kappa statistic.

31. The database of claim 26, wherein the comparison context comprises a description of the b-tree organization.

32. The database of claim 31, wherein the b-tree organization comprises a hierarchical organization of a plurality of levels, each level corresponding to an attribute selected for filtering the first plurality of records.

33. The database of claim 26, further comprising a fifth plurality of records comprising manually-generated comparisons, wherein the fourth plurality of records further comprises a link for associating the fifth plurality of records with the first, second and third pluralities of records.

34. The database of claim 26, wherein the first plurality of records further includes a regulation event identifier associated with each gene regulation event.

35. The database of claim 26, wherein the first, second, third, fourth and fifth pluralities of records are arranged in a relational format comprising a plurality of relational tables.

36. The database of claim 35 wherein the plurality of relational tables comprises at least an event table, a comparison table, a control vocabulary table, a context table, a sample table, a manually-generated comparison table and a gene family table.
